(19) United States
(12) Patent Application Publication
(10) Pub. No.: US 2023/0233467 A1
(43) Pub. Date: Jul. 27, 2023

(54) MULTIPLE NUTRIENT LIPOSOMAL SUPPLEMENT AND METHODS OF MANUFACTURING THE SAME

(71) Applicant: Nutraceutical Corporation, Salt Lake City, UT (US)

(72) Inventors: Max F. Willis, Salt Lake City, UT (US); Neil C. Roberts, Salt Lake City, UT (US)

(73) Assignee: Nutraceutical Corporation, Salt Lake City, UT (US)

(21) Appl. No.: 18/159,030

(22) Filed: Jan. 24, 2023

Related U.S. Application Data
(60) Provisional application No. 63/302,503, filed on Jan. 24, 2022.

(51) Int. Cl. A61K 9/127; A61K 35/60; A61K 33/26; A61K 31/728; A61K 36/81; A61K 31/198; A61K 36/42; A61K 36/45; A61K 31/122; A61K 31/07; A61K 31/375; A61K 31/355; A61K 31/4415; A61K 31/714; A61K 31/525; A61K 31/455; A61K 31/519; A61K 31/4188; A61K 31/14; A61K 33/32; A61K 33/30; A61K 33/24; A61K 33/06; A61K 33/04; A61K 47/24

(52) U.S. Cl. CPC A61K 9/127; A61K 35/60; A61K 33/26; A61K 31/728; A61K 36/81; A61K 31/198; A61K 36/42; A61K 36/45; A61K 31/122; A61K 31/07; A61K 31/375; A61K 31/355; A61K 31/4415; A61K 31/714; A61K 31/525; A61K 31/455; A61K 31/519; A61K 31/4188; A61K 31/14; A61K 33/32; A61K 33/30; A61K 33/24; A61K 33/06; A61K 33/04; A61K 47/24

(57) ABSTRACT

Compositions and methods for increasing absorption of nutrients by a body. A composition includes a liposome comprising a lipid bilayer, wherein the liposome comprises an internal core. The composition includes a nutrient payload disposed within the liposome. The nutrient payload comprises a plurality of different supplement nutrients comprising one or more of a vitamin, mineral, extract, enzyme, or amino acid.

### TECHNICAL FIELD

The disclosure relates generally to compositions of matter and particularly relates to liposomal compositions.

### BACKGROUND

Many people seek to consume vitamins, mineral, enzymes, plant extracts, amino acids, herbs, and other specialty supplements such as probiotics, fish oil, and digestive enzymes, as part of an everyday self-care regimen. The use of nutrient supplements has accelerated over the past several years and is expected to continue to increase and retain in the long term.

Multivitamins lead the way as the largest segment of all supplements and are normally the first entry point into the supplement category. Those who consume a daily multivitamin look to optimize their vitamin intake where the standard diet is deficient. These persons seek a convenient, whole-body wellness solution with antioxidant support, sustained energy levels, immune support, and more.

Vitamin, mineral, herb, and other supplements can provide numerous benefits to the body. Supplements are intended to be a cost effective, and timesaving means to deliver essential nutrients to the body. However, it is estimated that the body absorbs less than ten percent of the nutrients in traditional tablets or capsules. Traditional nutrient delivery systems are deficient in numerous ways and fail to enable the body to absorb any significant percentage of vitamins, minerals, herbs, or other supplements.

The traditional nutrient delivery systems break down when faced with digestive enzymes and stomach acids within the gastrointestinal system. This significantly reduces the ratio of nutrients that are available to be absorbed by the body and provide the intended benefit. Further, foods comprise “anti-nutrients” or cause the body to generate anti-nutrients that reduce the percentage of nutrients that are absorbed into the bloodstream. These anti-nutrients bind to the delivered nutrients through enzymatic action or fermentation. Some common anti-nutrients include lectins, oxalates, and phytates. Further, processed, and cooked foods lack essential enzymes, and fortified foods include synthetic vitamins that are difficult to assimilate into the food.

The malabsorption paradigm presents an opportunity for innovation within the nutrient supplementation space. Over the years, supplement manufacturers have explored ways to offer greater absorption with technologies such as chelation, methylation, fermentation, delayed release, and more. While many of these advancements help to some degree, overall absorption remains a concern. What is needed are improved compositions, methods, and systems for delivering nutrients to a body such that the nutrients can be absorbed and used by the body.

Considering the foregoing, disclosed herein are compositions and methods for improved nutrient delivery systems for delivering nutrients to a body.

### BRIEF DESCRIPTION OF THE DRAWINGS

Non-limiting and non-exhaustive implementations of the present disclosure are described with reference to the following figures, wherein like reference numerals refer to like parts throughout the various views unless otherwise specified. Advantages of the present disclosure will become better understood with regard to the following description and accompanying drawings where:

FIG. 1 is a perspective view of a schematic illustration of a cross-section of a liposome comprising a lipid bilayer and an internal core;

FIG. 2 is a straight-on side view of a schematic illustration of a cross-section of a lipid bilayer;

FIG. 3 is a straight-on view of a cross-section of a liposome comprising a lipid bilayer and an internal core, wherein a payload comprising a plurality of different nutrients may be disposed within the internal core and/or the payload may be disposed anywhere within the liposome including within the lipid bilayer;

FIG. 4 is an image captured by Dr. Belnap at the University of Utah using a Transmission Electron Microscope (TEM) that depicts a plurality of liposomes that each comprise a nutrient payload disposed therein;

FIG. 5 is an image captured by Dr. Belnap at the University of Utah using a TEM that depicts a liposome comprising a nutrient payload disposed therein;

FIG. 6 is an image captured by Dr. Belnap at the University of Utah using a TEM that depicts a liposome comprising a nutrient payload disposed therein;

FIG. 7 is a graph illustrating the unexpectedly good results for absorption of Vitamin C when delivered by a liposomal delivery system versus a convention non-liposomal delivery system; and

FIG. 8 is a schematic flow chart diagram of a method for preparing a liposomal nutrient supplement and providing the liposomal nutrient supplement to a user.

### DETAILED DESCRIPTION

Disclosed herein are compositions, methods, and delivery systems for providing vitamins, minerals, extracts, amino acids, and other nutrients to a body. The compositions, methods, and delivery systems described herein include multiple nutrient compositions encased in a liposomal shell comprised of phospholipid bilayers. Specifically disclosed herein are liposomal nutrient supplements for oral delivery to a person or animal. The liposomal nutrient supplements described herein exhibit increased nutrient absorption in a body when compared with conventional non-liposomal supplements.

A composition described herein includes a liposome comprising a lipid bilayer, wherein the lipid bilayer comprises a spherical geometry disposed around an internal core. The composition comprises a payload disposed within the internal core defined by the lipid bilayer of the liposome and/or the payload may be disposed anywhere within the liposome including within the lipid bilayer. The payload comprise water and a plurality of different nutrients comprising one or more of vitamins, minerals, plant extracts, or amino acids. The liposome comprising the payload is a liposomal nutrient supplement for efficient delivery of vitamins, minerals, plant extracts, and/or amino acids to a user.

The liposomal compositions described herein exhibit unexpectedly good results when compared with other liposomal formations. With traditional liposomal fabrication techniques, it is difficult to load a liposomal bilayer with a complex and large payload comprising multiple different nutrients. Specifically, traditional liposomal fabrication techniques have not successfully manufactured a multivitamin within a liposomal shell.

Additionally, the multiple nutrient liposomal supplements described herein exhibit unexpectedly good results when compared with conventional non-liposomal delivery systems and further when compared with single-nutrient liposomal delivery systems. The multiple nutrient liposomal supplements described herein exhibit increased absorption by a body when compared with conventional non-liposomal nutrient delivery systems such as tablets, capsules, and powders. Additionally, the multiple nutrient liposomal supplements described herein exhibit unexpectedly good results for maintaining liposomal structure, withstanding the harsh conditions of the gastrointestinal tract, and delivering multiple different nutrients to a body's bloodstream, when compared with single-nutrient liposomal delivery systems.

The multiple nutrient liposomal delivery systems described herein overcome numerous challenges that are traditionally associated with liposomal delivery systems. In most cases, a liposomal supplement provides a single nutrient to a user. In these cases, the liposomal shell encases a single type of vitamin, mineral, or another nutrient. This is because it is challenging to prepare a multiple nutrient liposomal supplement, wherein the payload disposed within the lipid bilayer comprises a plurality of different nutrients. The conventional liposomal supplements (wherein the payload includes a single type of nutrient) are simpler to prepare because the ratio of nutrients (e.g., vitamins, minerals, amino acids) to fats (i.e., lipids used for preparing the lipid bilayer of the liposome) is closer. However, when preparing a multiple nutrient liposomal supplement, the ingredients typically comprise about 30% fats (for preparing the lipid bilayer) and 70% nutrients for the payload disposed within the liposome. In this case, it is challenging to ensure that the liposomal structure remains intact, the liposomal structure can withstand the harsh conditions of the gastrointestinal tract, and the liposomal structure can successfully deliver the payload into a cell within the body. The multiple nutrient liposomal supplements described herein overcome these challenges and exhibit unexpectedly good results when compared with single-nutrient liposomal supplements.

Described herein is a grouping of multiple vitamins, minerals, extracts, amino acids, and/or other nutrients that are encased in a liposomal shell. The liposome forms a phospholipid bilayer that encases water and fat-soluble nutrients. This protects the nutrients like a shield from the rigors of digestion. A liposomal nutrient supplement is constructed of three ingredients, including the phospholipid bilayer, water, and a payload of nutrients. This enables the nutrients to travel safely and reach places where nutrients typically have difficulty reaching. The liposomal shells therefore protect the fragile nutrients to support greater absorption into the bloodstream and throughout the body.

The compositions and methods described herein enable increased uptake of vitamins, minerals, plant extracts, amino acids, and other nutrients by the body. Traditional non-liposomal vitamin delivery enables less than 10% of the nutrients to be absorbed by the body. The nutrients stored within the tablet are broken down by the harsh environment within the gastrointestinal tract before the nutrients can be absorbed into the bloodstream. Additionally, natural “anti-nutrients” like lectins and oxalates in food and food-based multivitamins/minerals interfere with the body's ability to absorb nutrients. The liposomal delivery compositions described herein overcome these challenges and enable increased absorption by the body of vitamins, minerals, and other nutrients.

The liposomal delivery systems described herein enable numerous further benefits over traditional non-liposomal delivery systems. The liposomal delivery system encases the nutrients and may mask the odors that may be associated with vitamins, minerals, and extracts. The liposomal delivery system protects the nutrients from stomach acid and harsh digestive enzymes. The liposomal delivery system disguises the nutrients by micronizing and masking the active ingredients to enable greater amounts of nutrients to be carried through the small intestine. The liposomal delivery system enables gentle digestion such that the vitamins, minerals, and other nutrients can be taken without food.

The liposomes described herein may be composed of naturally derived phospholipids with mixed lipid chains, such as egg phosphatidylethanolamine, or of pure surfactant components like dioleoylphosphatidylethanolamine (DOP). Liposomes may further comprise a core aqueous solution that is trapped by one or more bilayers. The phospholipid bilayers of a liposome may originate from natural sources, which are biologically inert, immunogenic, and exhibit a lower inherent toxicity. The liposomes described herein may vary in size from 0.025 micrometers up to 2.5 micrometers.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Before the structures, systems, methods, and compositions for activating cellular stress signaling pathways are disclosed and described, it is to be understood that this disclosure is not limited to the structures, configurations, process steps, and materials disclosed herein as such structures, configurations, process steps, and materials may vary somewhat. It is also to be understood that the terminology employed herein is used for the purpose of describing embodiments only and is not intended to be limiting since the scope of the disclosure will be limited only by the appended claims and equivalents thereof.

In describing and claiming the subject matter of the disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified ingredients, materials, or steps and those that do not materially affect the basic and novel characteristic or characteristics of the claimed disclosure.

As used herein, “effective amount” means an amount of an ingredient or a component of the product that is nontoxic, but sufficient to provide the desired effect and performance at a reasonable benefit/risk ratio attending any dietary supplement or product. For example, an effective amount of a vitamin or mineral is an amount sufficient to prevent a deficiency thereof and to reduce the incidence of some adverse effects.

As used herein, a component or ingredient of a composition may include any suitable form of the component or ingredient, such as, for example, an extract, a powder, a tincture, an absolute, an essential oil, a paste, a dehydrated form, and so forth. It should be appreciated that any suitable form or combination of a component or ingredient may be used unless otherwise specified.

As used herein, an “extract” includes any substance obtained from a raw material. An extract may be obtained from a blossom, fruit, root, nut, whole plant, leaf, or other component of an agricultural or horticultural component. An extraction may be obtained through expression, absorption, maceration, distillation, grinding, dehydration, and so forth. An extract may be stored in a solvent such as ethanol or water or may be stored in a dry form such as a powder. As an example, a plant extract may comprise a portion of an herb or other plant and may comprise a ground or dehydrated herb or an extract obtained through expression, absorption, maceration, distillation, or other means.

As used herein, a “supplement nutrient” includes any substance, molecule, or compound that is intended to provide nutrients to a body. By way of example only, a supplement nutrient may comprise any of a vitamin, mineral, herb, enzyme, acid, amino acid, extract, fish oils, probiotics or specialty nutrients such as Coenzyme Q-10.

Unless defined otherwise, all technical and scientific terms used herein have the same meanings as commonly understood by one of ordinary skill in the art to which this disclosure pertains and belongs.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to embodiments are not restricted to only those embodiments in which they are described. For example, an element described in reference to one embodiment or figure, may be alternatively included in another embodiment or figure regardless of whether those elements are shown or described in another embodiment or figure. In other words, elements in the figures may be interchangeable between various embodiments disclosed herein, whether shown or not.

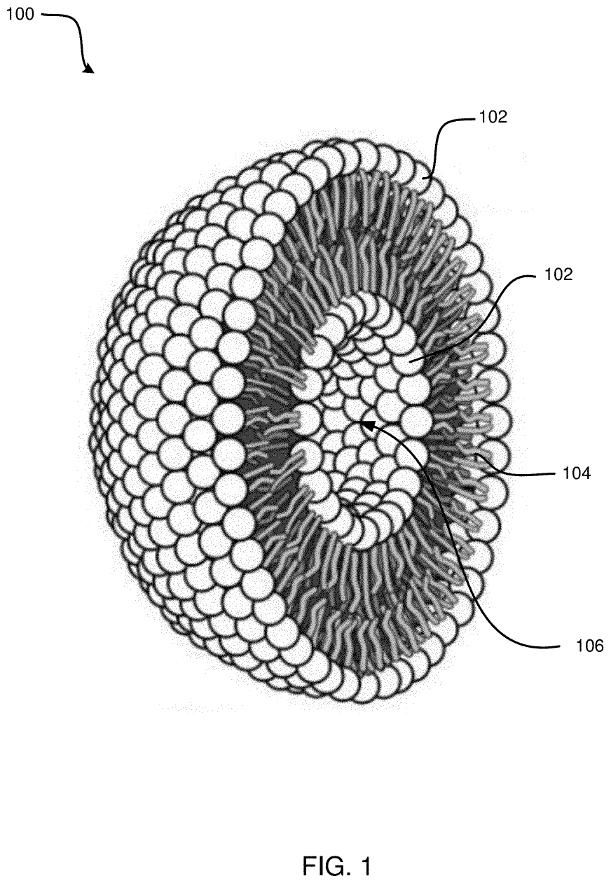

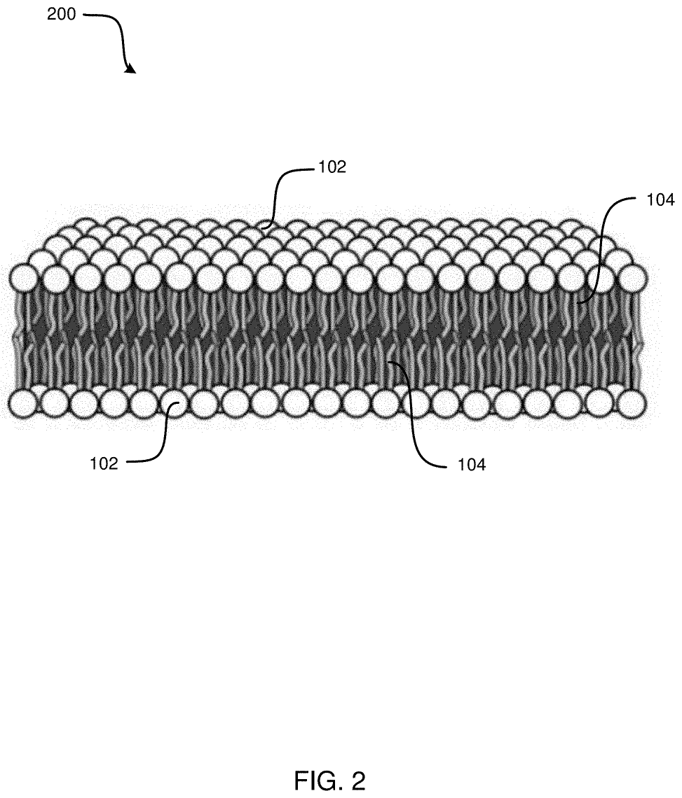

Referring now to the figures, FIG. 1 is a cross-sectional perspective view of one-half of a liposome 100. The liposome 100 comprises a plurality of phospholipid heads 102 (may also be referred to as lipid heads) and further comprises a plurality of phospholipid tails 104 (may also be referred to as lipid tails). The liposome 100 illustrated in FIG. 1 comprises only one phospholipid bilayer. In alternative embodiments, the liposome 100 may comprise a plurality of phospholipid bilayers layered upon each other around an internal core 106. FIG. 2 is a cross-sectional side view of a phospholipid bilayer 200. The phospholipid bilayer 200 forms the liposome 100 structure illustrated in FIG. 1.

The compositions and methods described herein implement liposomal delivery systems. A liposome 100 is a vesicle composed of one or more lipid bilayer 200s. The liposomal shell may comprise a spherical shape and closely resemble the structure of a cell membrane, as illustrated in FIG. 1. The phospholipid bilayer 200 of the liposome 100 comprises the phospholipid heads 102, which are hydrophilic, or “water loving” groups. The phospholipid bilayer 200 of the liposome 100 further comprises phospholipid tails 104, which are lipophilic, or “fat loving” groups composed of a long hydrocarbon chain that repels water. When the phospholipid bilayer 200 membrane is exposed to water, the phospholipid heads 102 are attracted to water and forms a surface that faces the water. At the same time, the phospholipid tails 104 are repelled by water and form a surface that opposes the water. Within a single phospholipid bilayer 200, one layer of the phospholipid heads 102 faces the exterior of the liposome, and another layer of the phospholipid heads 102 faces the internal liposomal environment. The hydrocarbon phospholipid tails 104 that are attached to both layers of phospholipid heads 102 face each other and form the bilayer 200 structure of the liposome 100.

The phospholipids comprise phosphatidylserine and phosphatidylcholine. The phospholipids are naturally occurring in lecithin, meat, egg yolks, seed oils, algae, and fish. In a particular embodiment described herein, the phospholipids within the phospholipid bilayer 200 are extracted from sunflower lecithin.

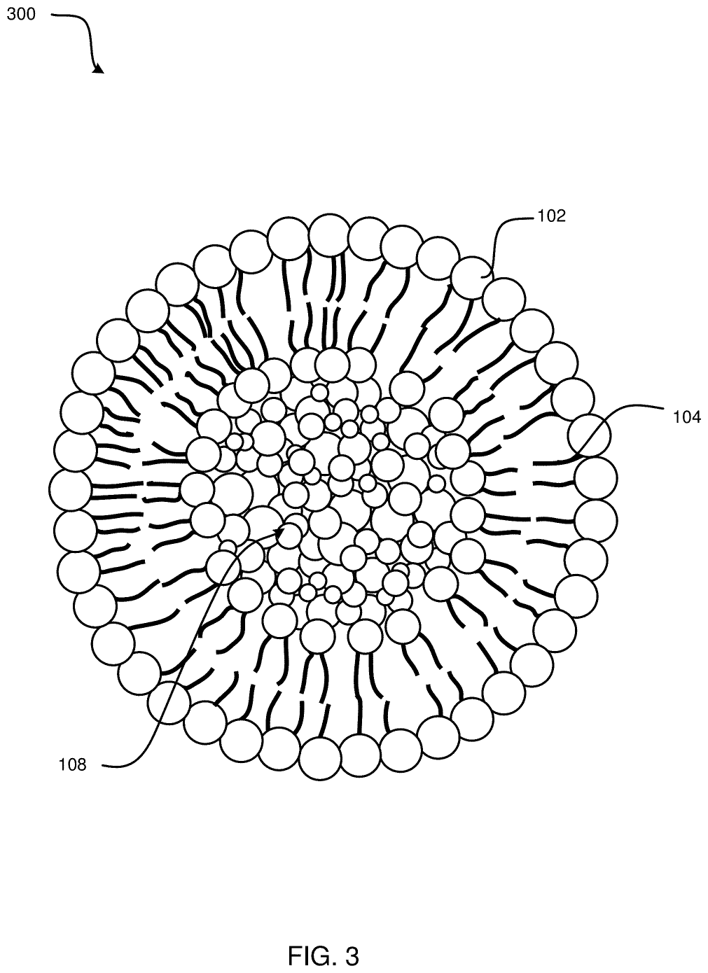

The phospholipid bilayer 200 forms a spherical or near-spherical shape that surrounds an internal core 106. The internal core 106 may comprise water and may further comprise a payload or portion of a payload. In an implementation, a multivitamin payload may be disposed within the internal core 106 of the liposome 100. In an implementation, a multivitamin payload may be disposed anywhere within the liposome including the one or more lipid bilayers. FIG. 3 illustrates a cross-sectional side view of a liposomal multivitamin 300. The liposomal multivitamin 300 comprises a liposome structure surrounding an internal core 106 and comprises a payload 108. The payload 108 comprises a plurality of different nutrients, wherein the nutrients comprise one or more of vitamins, minerals, enzymes, plant extracts, fungi extracts, amino acids, and other nutrients.

In an implementation, the payload 108 comprises one or more of: Vitamin A, Vitamin C, Vitamin D-3, Vitamin E, Vitamin K-1, Vitamin K-2, Thiamine, Riboflavin, Niacin, Vitamin B-6, Folate, Vitamin B-12, Biotin, Pantothenic Acid, Choline, Iron, Iodine, Magnesium, Zinc, Selenium, Manganese, Chromium, Molybdenum, Glycine, Inositol, PABA, Liposomal Trace Mineral Complex (may include a blend of 72+ trace minerals from inland seabed), Lutein, Coenzyme Q10, Rose Hips, Acerola, Citrus Bioflavonoids, Silica, Aloe, Ginger, Ashwagandha, GABA, Hyaluronic Acid, L-Leucine, L-Isoleucine, L-Valine, L-Glutamine, Pumpkin extract, and so forth. The multivitamin payload may comprise any suitable combination of vitamins, minerals, herbs, and other dietary supplements as deemed suitable for the application.

In some implementations, the payload 108 is prepared specifically for liposomal delivery. In these implementations, the following vitamins, minerals, herbs, and other supplements may be prepared in the following forms. Vitamin A may be prepared as liposomal beta carotene, Vitamin C may be prepared as liposomal ascorbic acid, Vitamin D-3 may be prepared as liposomal cholecalciferol from algae, Vitamin E may be prepared as liposomal d-alpha tocopherol from sunflower oil, Vitamin K-1 may be prepared as liposomal phylloquinone, Vitamin K-2 may be prepared as liposomal menaquinone-7 (MK-7), thiamine may be prepared as liposomal thiamine or liposomal benfotiamine, riboflavin may be prepared as liposomal riboflavin or liposomal riboflavin-5-phosphate, niacin may be prepared as liposomal niacin or liposomal niacinamide, Vitamin B-6 may be prepared as liposomal pyridoxine or liposomal pyridoxal-5-phosphate, folate may be prepared from liposomal folic acid or liposomal 6S-5-methyltetrahydrofoloic acid and glucosamine salt, Vitamin B-12 may be prepared as liposomal cyanocobalamin or liposomal methylcobalamin, biotin may be prepared as liposomal biotin, pantothenic acid may be prepared from liposomal calcium d-pantothenate, choline may be prepared from liposomal choline bitartrate, iron may be prepared from liposomal ferrous fumarate, iodine may be prepared from liposomal potassium iodide, magnesium may be prepared from liposomal magnesium oxide, including a variety of chelations such as citrate, glycinate, taurate or malate, zinc may be prepared from liposomal zinc oxide, including a variety of chelations such as citrate, selenium may be prepared as liposomal yeast-free l-selenomethionine, manganese may be prepared from liposomal manganese citrate, chromium may be prepared from liposomal chromium picolinate, molybdenum may be prepared from liposomal molybdenum citrate, glycine may be prepared from liposomal glycine, inositol may be prepared as liposomal inositol, PABA may be prepared as liposomal PABA, lutein may be prepared from liposomal lutein from marigold, coenzyme Q-10 may be prepared as liposomal coenzyme Q-10, rose hips may be prepared as liposomal rose hips, acerola may be prepared as liposomal acerola, and so forth.

The payload 108 may be disposed within the internal core 106 that is defined by the phospholipid bilayer 200 of the liposome 100 and/or the payload may be disposed anywhere within the liposome 100 including the phospholipid bilayer 200. The payload 108 comprises a plurality of unique vitamins, minerals, plant extracts, amino acids, and other nutrients. In an implementation, the payload 108 comprises a plurality of different vitamins. In an implementation, the payload 108 comprises one or more hydrophilic nutrient supplements and further comprises one or more hydrophobic nutrient supplements. The payload 108 further comprises an aqueous solution.

The liposomal delivery compositions described herein enable numerous benefits over non-liposomal delivery of vitamins and minerals. The liposomal delivery enables high bioavailability and absorption when compared with other oral forms of supplements. The liposomal delivery is noninvasive and avoids the pain or discomfort associated with injections, and further decreases the contamination risk associated with injections. The micronized encapsulation enabled by the liposomal shell protects against the harsh environment of the gastrointestinal tract and increases enteric or intestinal uptake and absorption. The liposomal delivery increases intracellular delivery of vitamins and minerals within the body.

The liposomes described herein may be manufactured using a variety of methods depending on the identity of the vitamins, minerals, extracts, amino acids, and other nutrients disposed therein. The choice of liposomal preparation methods may be optimized based on the following parameters. One parameter is based on the physiochemical characteristics of the material to be entrapped within the liposome. Another parameter is the nature of the medium in which the lipid vesicles are dispersed. Another parameter is the effective concentration of the entrapped substance and its potential toxicity. Another parameter is based on additional processes involved during application and delivery of the vesicle. Another parameter is based on the optimum size, polydispersity, and shelf life of the liposome for its intended application. A final parameter is based on batch-to-batch reproducibility and possibility of large-scale production of safe and efficient liposomal products. The liposomes described herein do not form spontaneously and are the product of supplying sufficient energy to a dispersion of phospholipids in a polar solvent, such as water, to break down multilamellar aggregates into oligo- or unilamellar bilayer vesicles.

The liposomes described herein may be manufactured using high-shear processing methods to break down particle size. The high-shear processing methods may include, for example, freeze-thaw cycles, rotor-stator dispersion, sonication, extrusion, and high-pressure homogenization. The small-sized liposomes described herein are shown to increase kinetics of absorption and lengthen circulation time within the body. The liposomes described herein may include phospholipids that are isolated from lecithins. The lecithins may be isolated from soybean, sunflower, canola, egg yolks, milk, and so forth.

The liposomes described herein serve as delivery vehicles for transporting substances into the body, and specifically for transporting a plurality of vitamins and/or mineral into the body. The liposomes facilitate absorption directly in the mouth or by preventing breakdown by stomach acid. The one or more lipid bilayers within the liposome creates a pocket of entrapped material and protects the entrapped material from hostile digestive conditions. This facilitates the uptake of the entrapped material by the gastrointestinal system.

The multiple nutrient liposomal supplements described herein may be provided to a user in a dehydrated form or an aqueous form. In an implementation, the multiple nutrient liposomal supplements described herein is dehydrated and provided to a user in a powdered form. The powdered liposomal supplement may be disposed within a capsule that is dissolvable within the user's digestive system. The powdered liposomal supplement may be compacted in a tablet or other form. The powdered liposomal supplement may be provided as a loose powder. The powdered liposomal supplement may be prepared for topical delivery by combining with one or more fluids to prepare a vitamin delivery emulsion. The powdered liposomal supplement may be mixed in with water or another liquid for ingestion by the user. The powdered liposomal supplement may be disposed within a liquid gel capsule and combined with an oil or other fluid. In another implementation, the multiple nutrient liposomal supplements described herein is provided to a user in an aqueous form. The aqueous form may be consumed orally, applied topically, delivered intravenously, and/or delivered intramuscularly.

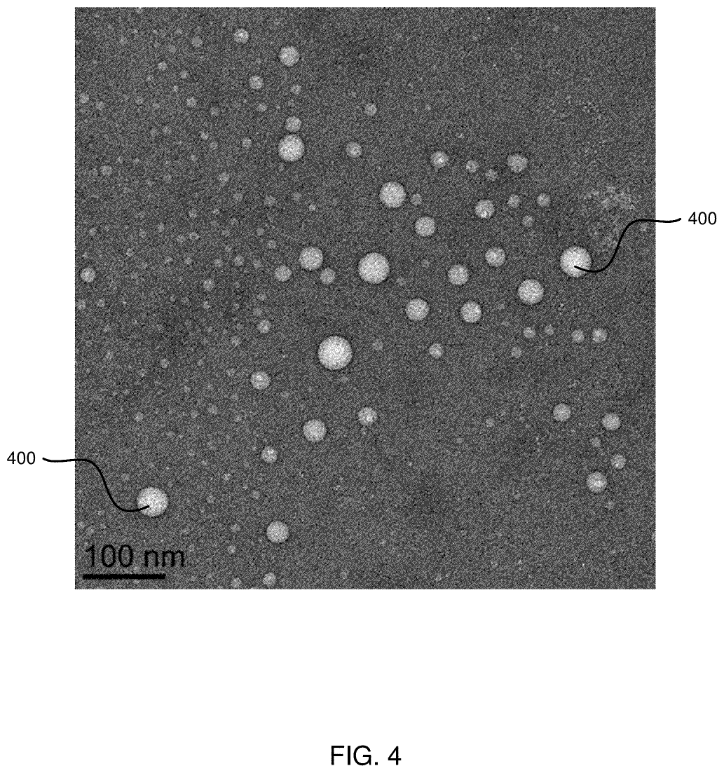

FIG. 4 illustrates an image depicting a plurality of liposomes and liposomal nutrient supplements as discussed herein. The image is captured under a Transmission Electron Microscope (TEM) and depicts dehydrated liposomal nutrient supplements. In the TEM capture, the liposomes 400 are shown as white circles against a black background. The liposomes 400 comprise spherical geometries. Each liposome 400 comprises a plurality of nutrients disposed therein, including vitamins, minerals, herbs, and other nutrients. As shown in the TEM capture, the liposomes 400 may be considered nanoliposomes and have a diameter less than 100 nm. In various implementations, it is desirable to reduce the size of the liposomal nutrient supplements to a nanoliposome size to ensure increased uptake and absorption by the body. The liposomes described herein may comprise a diameter from about 10 nm to about 3 μm. The liposomal nutrient supplements described herein, including those illustrated in FIGS. 4-6, are dehydrated prior to delivery to a user.

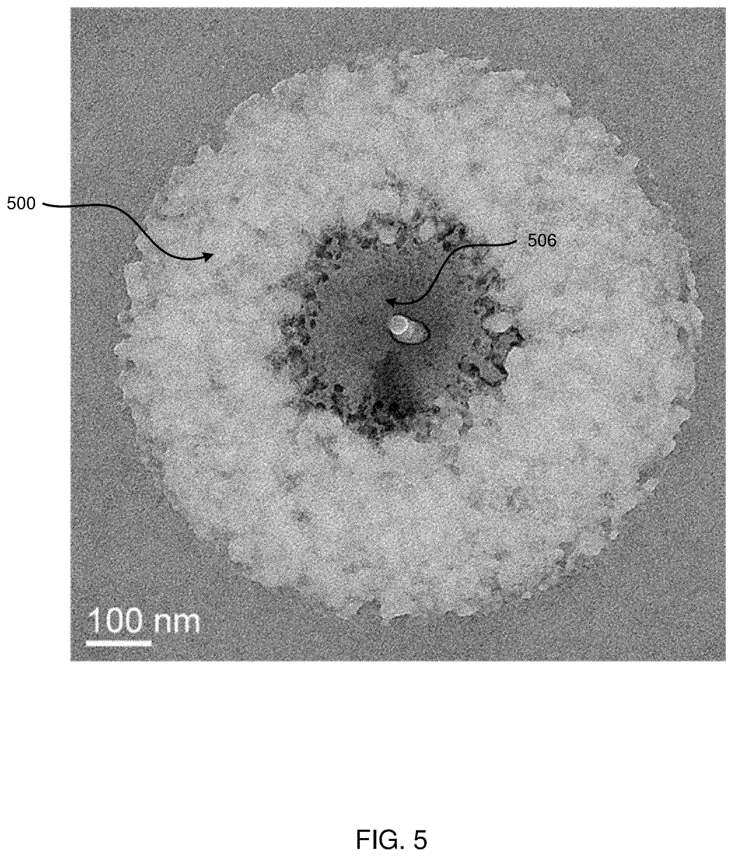

FIG. 5 illustrates an image depicting a liposomal multivitamin as discussed herein. The image is captured under a Transmission Electron Microscope (TEM) and depicts a dehydrated liposomal nutrient supplement. As shown in FIG. 5, the liposomal multivitamin comprises a spherical or near-spherical shape, wherein the spherical shape is constructed of a phospholipid bilayer 500 protecting an internal core 506. A plurality of nutrients may be disposed within the internal core 506 and/or the plurality of nutrients may be disposed anywhere within the liposome including the phospholipid bilayer. The TEM capture provides a two-dimensional approximation of the spherical geometry of the liposomal nutrient supplement.

The TEM image shown in FIG. 5 illustrates that the techniques described herein have been successfully deployed to generate a liposomal structure comprising a multivitamin payload. This indicates unexpectedly good results when compared against traditional systems, wherein only one vitamin or nutrient (e.g., Vitamin C in many cases) is stored within a liposomal bilayer.

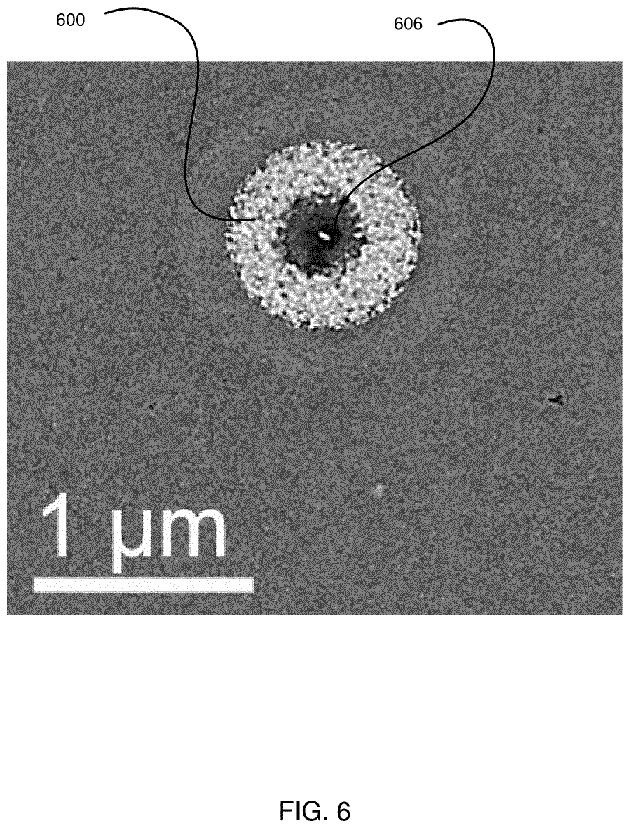

FIG. 6 illustrates an image depicting a liposomal multivitamin as discussed herein. The image is captured under a Transmission Electron Microscope (TEM) and depicts a dehydrated liposomal nutrient supplement. As shown in FIG. 6, the liposomal multivitamin comprises a spherical or near-spherical shape, wherein the spherical shape is constructed of a phospholipid bilayer 600 protecting an internal core 606. The internal core 606 may comprise a plurality of different vitamins, minerals, herbs, or other nutrient supplements. It will be appreciated that the plurality of different vitamins, minerals, herbs, or other nutrient supplements may be disposed anywhere within the liposome including the phospholipid bilayer 600. The liposomal nutrient supplement illustrated in FIG. 6 comprises a larger diameter when compared with the liposomal nutrient supplements depicted in FIGS. 4-5. It should be appreciated that the geometries of the liposomal nutrient supplements can vary depending on the manufacturing processes, the substances disposed within the payload, and so forth. Various applications may call for different dimensions for the liposomal structure.

The liposomal nutrient supplements illustrated in FIGS. 4-6 are prepared and dehydrated prior to delivery to a user. A method includes preparing a liposomal nutrient supplement that comprises a phospholipid bilayer and a plurality of different vitamins, minerals, herbs, or other nutrients. The liposomal nutrient supplement is dehydrated. The dehydrated liposomal delivery system is more stable and has decreased water activity. Additionally, the dehydrated liposomal delivery system has improved resistance to the rigors of digestion. When the dehydrated liposomal nutrient supplement reaches the user's small intestine, the dried liposome is rehydrated and absorbed into the active transport site of the bowel. These liposomes then pass more easily into the blood for transport throughout the body to deliver maximum support of the user.

The liposomal nutrient supplements described herein are manufactured without added chemicals, heat, or pressure. The liposomal delivery system is gentle on the gastrointestinal system, may mask some of the typical smells that may be associated with the nutrients within the payload, and does not cause gas or nausea.

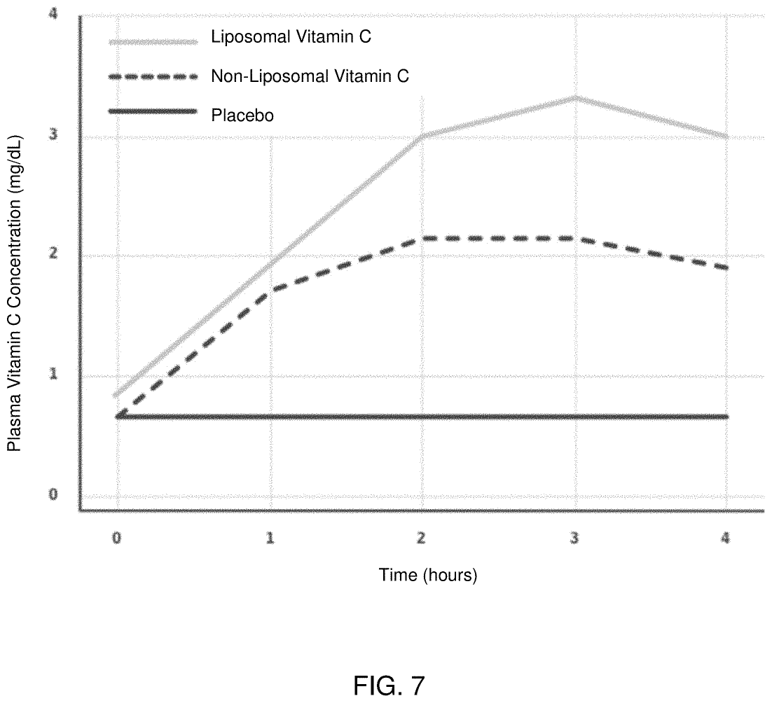

FIG. 7 illustrates a graph depicting the increased uptake by a body of Vitamin C that is delivered in a liposomal delivery system versus Vitamin C that is delivered in a non-liposomal delivery system. The uptake of the body is measured based on plasma Vitamin C concentration in milligrams per deciliter. As shown in FIG. 7, the liposomal Vitamin C delivery system enables significantly increased absorption by the body when compared with the non-liposomal Vitamin C delivery system.

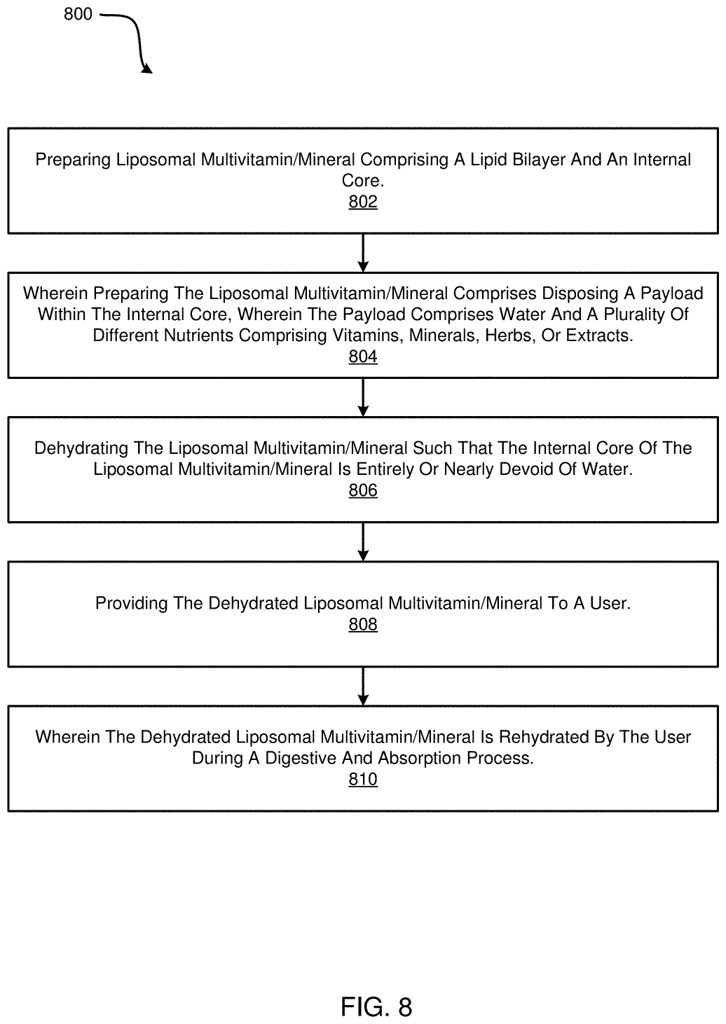

FIG. 8 is a schematic flow chart diagram of a method 800 for preparing and providing a liposomal nutrient supplement. The method 800 includes preparing at 802 the liposomal nutrient supplement comprising a lipid bilayer and an internal core. The method 800 is such that preparing the liposomal nutrient supplement comprises disposing a payload within the internal core and/or disposing the payload anywhere within the liposome including the phospholipid bilayer, wherein the payload comprises water and a plurality of different nutrients comprising vitamins, minerals, herbs, extracts, or other nutrients (see 804). The payload comprises at least two unique nutrients such that the multiple different types of nutrients are disposed within the liposomal nutrient supplement. The payload may comprise a combination of vitamins, minerals, plant extracts, fatty acids, and other nutrients. The ingredients of the payload are optimized depending on the needs of the end-user.

The method 800 includes dehydrating at 806 the liposomal nutrient supplement such that the internal core of the liposomal nutrient supplement is entirely or nearly devoid of water. In an embodiment, the payload disposed within the internal core comprises from about 0.3% to about 3%, and maybe between about 0.8% to about 2% water when dehydrated. The process of dehydrating at 806 includes drawing out the water and other liquids from the liposomal nutrient supplement. The lipid bilayer of the liposome will then collapse on itself and form a dehydrated composition that comprises the lipid bilayer and the payload. The liposomal nutrient supplement may be prepared in a powdered form following the dehydration process.

The method 800 includes providing at 808 the dehydrated liposomal nutrient supplement to a user. The dehydrated liposomal nutrient supplement may be provided in a powdered form, and the powdered substance may be disposed within a capsule that will be dissolved by the user's body. The method 810 is such that the dehydrated liposomal nutrient supplement is rehydrated by the user during a digestive and/or absorption process (see 810). Specifically, the liposomal delivery system may be configured to remain dehydrated until it encounters the user's digestive tract and is then rehydrated prior to binding with cells within the user's small intestine. The contents of the payload may then be absorbed into cells within the user's digestive tract and then taken up by the user's bloodstream for distribution around the user's body.

The liposomal delivery systems described herein show increased efficacy for delivering a plurality of different vitamins, minerals, or other nutrient supplements to a body when compared with traditional USP vitamins and minerals (non-liposomal) in capsule, tablet, powder, softgel, lozenge or liquid form. Studies indicate that the liposomal nutrient supplement described herein shows greater appearance of select vitamins and mineral in plasma or serum following intake when compared with a traditional multivitamin/mineral. A traditional multivitamin/mineral is produced by a conventional process of blending nutrients together and filling them into a hard-shelled capsule. The liposomal nutrient supplement described herein includes generating a liposomal protective bilayer and filling the liposome, including the lipid bilayers, and/or the internal core 106 of the liposome 100 with a payload comprising a plurality of multivitamins/minerals.

The liposomal nutrient supplement exhibits unexpectedly good results at delivering each of a plurality of different vitamins, minerals, herbs, and other nutrient supplements to a body. The plasma and serum vitamin and mineral levels are greater after intake of the liposome-based formula when compared with conventional USP or food based formulas.

The liposomal nutrient supplement described herein serves as a protective vehicle for the nutrients within the payload. The payload comprises a plurality of different vitamins, minerals, herbs, or other nutrient supplements. The liposomal shell helps protects the payload from gastric acid, digestive enzymes and other constituents which make up the harsh environment of the gastrointestinal system. When the active ingredients in the liposome is protected during digestion, the vitamins and minerals included therein remain intact and available for absorption into the body's bloodstream. The payload comprises water and a plurality of different water-soluble and fat-soluble vitamins, minerals, herbs, and other nutrients. The ingredients disposed within the liposomal-encased payload are more readily absorbed by the body.

The liposomal nutrient supplement described herein may be absorbed by a cell within a body. The phospholipid bilayer of the liposomal resembles the phospholipid bilayer of a cell wall within the body. Because the liposome is structurally like a cell wall, the phospholipid bilayer of the liposome may connect to the phospholipid bilayer of the cell wall, open, and release the payload into the cell. Further because the liposome resembles a cell wall, the phospholipid bilayer masks the ingredients within the payload so that larger quantities of the ingredients can be carried through the small intestine. When the liposomal shell is absorbed by the cell and integrated into the cell wall, the payload stored within the liposome is released into the cell. The payload is then absorbed by the body and can be transferred to other areas of the body through natural physiological processes.

Studies indicate that the liposomal delivery systems described herein lead to greater bioavailability of vitamins, minerals, herbs, and other nutrients. One study indicates that the body absorbs the lowest percentage of nutrients from food. The body absorbs three times as many nutrients from a conventional multivitamin/mineral when compared with food. The body absorbs nine times as many nutrients from a liposomal nutrient supplement as described herein, when compared with food. The body absorbs the greatest percentage of nutrients through intravenous or intramuscular administration. However, intravenous, and intramuscular administrations are expensive, time consuming, inconvenient, and can introduce infectious organisms to the body. The liposomal delivery systems described herein provide the most effective means for providing vitamins, minerals, herbs, and other nutrients to a body.

The identity and relative mass percentages of the ingredients within the payload are micronized and optimized in order to be enveloped by the liposome's phospholipid bilayers in order to become part of the lipospheric structure of said liposomes. The liposomal structure can withstand the harsh conditions of the gastrointestinal tract, and the liposomal structure can successfully attach to a cell wall to deliver the payload directly to a cell. The multiple nutrient liposomal delivery systems described herein exhibit unexpectedly good results when compared with liquid liposomal delivery systems. The examples below indicate optimized payload compositions for ensuring the success of the multiple nutrient liposomal delivery system.

Additionally, the identity and relative mass percentages of the ingredients within the payload are optimized to increase outcomes and effectuate a desire result in a user. In some cases, the payload is optimized for users with certain conditions, ages, sex, and so forth.

### Examples

The following examples pertain to further embodiments.

Table 1 below shows an example embodiment of the composition of a payload that is delivered within a liposomal delivery system when the payload is in a fully dehydrated form. In this example embodiment, the mass percentage of each component/ingredient is plus or minus five percent of the stated value.

TABLE 1

| | Mass Percentage of |
|---|---|
| Component | Total Payload (wt %) |
| Vitamin A | 0.3 |
| Vitamin C | 30.0 |
| Vitamin D-3 | 0.06 |
| Vitamin E | 5.0 |
| Vitamin K-1 | 0.04 |
| Thiamine | 0.4 |
| Riboflavin | 0.4 |
| Niacin | 5.3 |
| Vitamin B-6 | 0.6 |
| Folate | 0.1 |
| Vitamin B-12 | 0.3 |
| Biotin | 0.09 |
| Pantothenic Acid | 1.7 |
| Choline | 6.6 |
| Iron | 3.0 |
| Iodine | 0.05 |
| Magnesium | 6.6 |
| Zinc | 3.6 |
| Selenium | 0.2 |
| Manganese | 0.8 |
| Chromium | 0.1 |
| Molybdenum | 0.1 |
| Glycine | 19.9 |
| Inositol | 8.3 |
| PABA | 1.7 |
| Liposomal trace mineral complex | 1.7 |
| Lutein | 0.3 |
| Coenzyme Q10 | 1.7 |
| Rose Hips | 0.7 |
| Acerola | 0.7 |

Table 2 below shows an example embodiment of the composition of a payload that is delivered within a liposomal delivery system when the payload is in a fully dehydrated form. In this example embodiment, the mass percentage of each component/ingredient is plus or minus five percent of the stated value.

TABLE 2

| | Mass Percentage of |
|---|---|
| Component | Total Payload (wt %) |
| Vitamin A | 0.3 |
| Vitamin C | 25.0 |
| Vitamin D-3 | 0.06 |
| Vitamin E | 4.2 |
| Vitamin K-1 | 0.03 |
| Thiamine | 0.3 |
| Riboflavin | 0.3 |
| Niacin | 4.5 |
| Vitamin B-6 | 0.5 |
| Folate | 0.1 |
| Vitamin B-12 | 0.3 |
| Biotin | 0.3 |
| Pantothenic Acid | 1.4 |
| Choline | 1.4 |
| Iron | 5.0 |
| Iodine | 0.04 |
| Magnesium | 5.6 |
| Zinc | 3.1 |
| Selenium | 0.2 |
| Manganese | 5.6 |
| Chromium | 0.1 |
| Molybdenum | 0.1 |
| Glycine | 16.7 |
| Inositol | 5.6 |
| PABA | 1.4 |
| Liposomal trace mineral complex | 1.4 |
| Lutein | 0.3 |
| Coenzyme Q10 | 1.4 |
| Rose Hips | 0.6 |
| Acerola | 0.6 |
| Citrus Bioflavonoids | 1.4 |
| Silica | 1.4 |
| Aloe | 1.4 |
| Ginger | 1.4 |
| Ashwagandha | 5.6 |
| GABA | 1.4 |
| Hyaluronic Acid | 1.4 |

Table 3 below shows an example embodiment of the composition of a payload that is delivered within a liposomal delivery system when the payload is in a fully dehydrated form. In this example embodiment, the mass percentage of each component/ingredient is plus or minus five percent of the stated value.

TABLE 3

| | Mass Percentage of |
|---|---|
| Component | Total Payload (wt %) |
| Vitamin A | 0.3 |
| Vitamin C | 25.1 |
| Vitamin D-3 | 0.06 |
| Vitamin E | 4.2 |
| Vitamin K-1 | 0.03 |
| Thiamine | 0.3 |
| Riboflavin | 0.4 |
| Niacin | 4.5 |
| Vitamin B-6 | 0.5 |
| Folate | 0.1 |
| Vitamin B-12 | 0.3 |
| Biotin | 0.08 |
| Pantothenic Acid | 1.4 |
| Choline | 1.4 |
| Iodine | 0.04 |
| Magnesium | 5.6 |
| Zinc | 3.1 |
| Selenium | 0.2 |
| Manganese | 0.6 |
| Chromium | 0.1 |
| Molybdenum | 0.1 |
| Glycine | 16.8 |
| Inositol | 7.0 |
| PABA | 1.4 |
| Liposomal trace mineral complex | 1.4 |
| Lutein | 0.3 |
| Coenzyme Q10 | 1.4 |
| Rose Hips | 0.6 |
| Acerola | 0.6 |
| L-Leucine | 7.0 |
| L-Isoleucine | 3.5 |
| L-Valine | 3.5 |
| L-Glutamine | 7.0 |
| Pumpkin | 1.4 |

Table 4 below shows an example embodiment of the composition of a payload that is delivered within a liposomal delivery system when the payload is in a fully dehydrated form.

TABLE 4

| | Mass Percentage of |
|---|---|
| Component | Total Payload (wt %) |
| Vitamin A | 0.3 ± 0.3 |
| Vitamin C | 30.0 ± 20 |
| Vitamin D-3 | 0.06 ± 0.04 |
| Vitamin E | 5.0 ± 4 |
| Vitamin K-1 | 0.04 ± 0.03 |
| Thiamine | 0.4 ± 0.2 |
| Riboflavin | 0.4 ± 0.2 |
| Niacin | 5.3 ± 3 |
| Vitamin B-6 | 0.6 ± 0.2 |
| Folate | 0.1 ± 0.1 |
| Vitamin B-12 | 0.3 ± 0.2 |
| Biotin | 0.09 ± 0.05 |
| Pantothenic Acid | 1.7 ± 0.5 |
| Choline | 6.6 ± 4 |
| Iron | 3.0 ± 2 |
| Iodine | 0.05 ± 0.03 |
| Magnesium | 6.6 ± 4 |
| Zinc | 3.6 ± 3 |
| Selenium | 0.2 ± 0.1 |
| Manganese | 0.8 ± 0.6 |
| Chromium | 0.1 ± 0.08 |
| Molybdenum | 0.1 ± 0.08 |
| Glycine | 19.9 + 10 |
| Inositol | 8.3 ± 6 |
| PABA | 1.7 ± 1 |
| Liposomal trace mineral complex | 1.7 ± 1 |
| Lutein | 0.3 ± 0.2 |
| Coenzyme Q10 | 1.7 ± 1 |
| Rose Hips | 0.7 ± 0.5 |
| Acerola | 0.7 ± 0.5 |

Table 5 below shows an example embodiment of the composition of a payload that is delivered within a liposomal delivery system when the payload is in a fully dehydrated form.

TABLE 5

| | Mass Percentage of |
|---|---|
| Component | Total Payload (wt %) |
| Vitamin A | 0.3 ± 0.2 |
| Vitamin C | 25.0 ± 20 |
| Vitamin D-3 | 0.06 ± 0.04 |
| Vitamin E | 4.2 ± 3 |
| Vitamin K-1 | 0.03 ± 0.02 |
| Thiamine | 0.3 ± 0.2 |
| Riboflavin | 0.3 ± 0.2 |
| Niacin | 4.5 ± 3 |
| Vitamin B-6 | 0.5 ± 0.2 |
| Folate | 0.1 ± 0.1 |
| Vitamin B-12 | 0.3 ± 0.2 |
| Biotin | 0.3 ± 0.2 |
| Pantothenic Acid | 1.4 ± 1 |
| Choline | 1.4 ± 1 |
| Iron | 5.0 ± 4 |
| Iodine | 0.04 ± 0.03 |
| Magnesium | 5.6 ± 4 |
| Zinc | 3.1 ± 2 |
| Selenium | 0.2 ± 0.2 |
| Manganese | 5.6 ± 4 |
| Chromium | 0.1 ± 0.1 |
| Molybdenum | 0.1 ± 0.1 |
| Glycine | 16.7 ± 10 |
| Inositol | 5.6 ± 4 |
| PABA | 1.4 ± 0.5 |
| Liposomal trace mineral complex | 1.4 ± 0.5 |
| Lutein | 0.3 ± 0.2 |
| Coenzyme Q10 | 1.4 ± 1 |
| Rose Hips | 0.6 ± 0.4 |
| Acerola | 0.6 ± 0.4 |
| Citrus Bioflavonoids | 1.4 ± 1 |
| Silica | 1.4 ± 1 |
| Aloe | 1.4 ± 1 |
| Ginger | 1.4 ± 1 |
| Ashwagandha | 5.6 ± 4 |
| GABA | 1.4 ± 1 |
| Hyaluronic Acid | 1.4 ± 1 |

Table 6 below shows an example embodiment of the composition of a payload that is delivered within a liposomal delivery system when the payload is in a fully dehydrated form.

TABLE 6

| | Mass Percentage of |
|---|---|
| Component | Total Payload (wt %) |
| Vitamin A | 0.3 ± 0.2 |
| Vitamin C | 25.1 ± 20 |
| Vitamin D-3 | 0.06 ± 0.04 |
| Vitamin E | 4.2 ± 3 |
| Vitamin K-1 | 0.03 ± 0.2 |
| Thiamine | 0.3 ± 0.2 |
| Riboflavin | 0.4 ± 0.2 |
| Niacin | 4.5 ± 4 |
| Vitamin B-6 | 0.5 ± 0.2 |
| Folate | 0.1 ± 0.1 |
| Vitamin B-12 | 0.3 ± 0.2 |
| Biotin | 0.08 ± 0.06 |
| Pantothenic Acid | 1.4 ± 1 |
| Choline | 1.4 ± 1 |
| Iodine | 0.04 ± 0.03 |
| Magnesium | 5.6 ± 4 |
| Zinc | 3.1 ± 2 |
| Selenium | 0.2 ± 0.1 |
| Manganese | 0.6 ± 0.4 |
| Chromium | 0.1 ± 0.1 |
| Molybdenum | 0.1 ± 0.1 |
| Glycine | 16.8 ± 10 |
| Inositol | 7.0 ± 5 |
| PABA | 1.4 ± 1 |
| Liposomal trace mineral complex | 1.4 ± 1 |
| Lutein | 0.3 ± 0.2 |
| Coenzyme Q10 | 1.4 ± 1 |
| Rose Hips | 0.6 ± 0.4 |
| Acerola | 0.6 ± 0.4 |
| L-Leucine | 7.0 ± 4 |
| L-Isoleucine | 3.5 ± 3 |
| L-Valine | 3.5 ± 3 |
| L-Glutamine | 7.0 ± 4 |
| Pumpkin extract | 1.4 ± 1 |

According to one or more embodiments of the disclosure, a composition may comprise one or more liposomes comprising a payload, wherein the payload comprises a combination of all or some, but not all, of the following ingredients:
a. Vitamin A (Beta Carotene, may be prepared without gelatin or sucrose);
b. Vitamin C (Ascorbic Acid);
c. Vitamin D-3 (may be extracted from algae);
d. Vitamin E (D-Alpha Tocopherol, may be extracted from sunflower with no soy);
e. Vitamin K-1 (Phylloquinone);
Thiamine (Thiamine or Benfotiamine);
g. Riboflavin (Riboflavin or Riboflavin-5-Phosphate);
h. Niacin;
i. Vitamin B-6 (Pyridoxine or Pyridoxine-5′-Phosphate);
j. Folate (Folic Acid or (6S)-5-Methyltetrahydrofolic Acid);
k. Vitamin B-12 (Cyanocobalamin or Methylcobalamin);
l. Biotin;
m. Pantothenic Acid;
n. Choline;
o. Iodine;
p. Magnesium;
q. Zinc;
r. Selenium;
s. Manganese;
t. Chromium;
u. Molybdenum;
v. Glycine;
w. Inositol;
x. PABA (Para-aminobenzoic acid, also known as Vitamin B-10);
y. Trace Mineral Complex;
z. Lutein;
aa. Coenzyme Q10;
bb. Rose Hips;
cc. Acerola;
dd. L-Leucine;
ee. L-Isoleucine;
ff. L-Valine;
gg. L-Glutamine;
hh. Pumpkin Seed Extract;
ii. Iron;
jj. Citrus Bioflavonoids;
kk. Silica;
ll. Aloe Extract;
mm. Ginger Extract;
nn. Ashwagandha Extract;
oo. GABA (Gamma-aminobutyric acid); or
pp. Hyaluronic Acid.

Embodiments of the composition may comprise one or more liposomes comprising a payload, wherein the payload comprises concentrations of Vitamin A as follows when the payload is in a fully dehydrated form:
a1) from about 0.1 wt % to about 2 wt % the total composition of the liposomal payload;
a2) from about 0.1 wt % to about 1.5 wt % the total composition of the liposomal payload;
a3) from about 0.1 wt % to about 1.0 wt % the total composition of the liposomal payload;
a4) from about 0.1 wt % to about 0.9 wt % the total composition of the liposomal payload;
a5) from about 0.1 wt % to about 0.8 wt % the total composition of the liposomal payload;
a6) from about 0.1 wt % to about 0.7 wt % the total composition of the liposomal payload;
a7) from about 0.1 wt % to about 0.6 wt % the total composition of the liposomal payload;
a8) from about 0.1 wt % to about 0.5 wt % the total composition of the liposomal payload; or
a9) from about 0.1 wt % to about 0.4 wt % the total composition of the liposomal payload.

Embodiments of the composition may comprise one or more liposomes comprising a payload, wherein the payload comprises concentrations of Vitamin C as follows when the payload is in a fully dehydrated form:
b1) from about 15 wt % to about 95 wt % the total composition of the liposomal payload;
b2) from about 20 wt % to about 95 wt % the total composition of the liposomal payload;
b3) from about 25 wt % to about 95 wt % the total composition of the liposomal payload;
b4) from about 30 wt % to about 95 wt % the total composition of the liposomal payload;
b5) from about 35 wt % to about 95 wt % the total composition of the liposomal payload;
b6) from about 40 wt % to about 95 wt % the total composition of the liposomal payload;
b7) from about 45 wt % to about 95 wt % the total composition of the liposomal payload;
b8) from about 50 wt % to about 95 wt % the total composition of the liposomal payload;
b9) from about 55 wt % to about 95 wt % the total composition of the liposomal payload;
b10) from about 60 wt % to about 95 wt % the total composition of the liposomal payload;
b11) from about 65 wt % to about 95 wt % the total composition of the liposomal payload;
b12) from about 70 wt % to about 95 wt % the total composition of the liposomal payload;
b13) from about 75 wt % to about 95 wt % the total composition of the liposomal payload;
b14) from about 80 wt % to about 95 wt % the total composition of the liposomal payload;
b15) from about 85 wt % to about 95 wt % the total composition of the liposomal payload;
b16) from about 90 wt % to about 95 wt % the total composition of the liposomal payload;
b17) from about 15 wt % to about 90 wt % the total composition of the liposomal payload;
b18) from about 15 wt % to about 85 wt % the total composition of the liposomal payload;
b19) from about 15 wt % to about 80 wt % the total composition of the liposomal payload;
b20) from about 15 wt % to about 75 wt % the total composition of the liposomal payload;
b21) from about 15 wt % to about 70 wt % the total composition of the liposomal payload;
b22) from about 15 wt % to about 65 wt % the total composition of the liposomal payload;
b23) from about 15 wt % to about 60 wt % the total composition of the liposomal payload;
b24) from about 15 wt % to about 55 wt % the total composition of the liposomal payload;
b25) from about 15 wt % to about 50 wt % the total composition of the liposomal payload;
b26) from about 15 wt % to about 45 wt % the total composition of the liposomal payload;
b27) from about 15 wt % to about 40 wt % the total composition of the liposomal payload;
b28) from about 15 wt % to about 35 wt % the total composition of the liposomal payload;
b29) from about 15 wt % to about 30 wt % the total composition of the liposomal payload;
b30) from about 20 wt % to about 35 wt % the total composition of the liposomal payload; or
b31) from about 22 wt % to about 32 wt % the total composition of the liposomal payload.

Embodiments of the composition may comprise one or more liposomes comprising a payload, wherein the payload comprises concentrations of Vitamin E as follows when the payload is in a fully dehydrated form:
d1) from about 2 wt % to about 10 wt % the total composition of the liposomal payload;
d2) from about 2 wt % to about 9 wt % the total composition of the liposomal payload;
d3) from about 2 wt % to about 8 wt % the total composition of the liposomal payload;
d4) from about 2 wt % to about 7 wt % the total composition of the liposomal payload;
d5) from about 2 wt % to about 6 wt % the total composition of the liposomal payload;
d6) from about 3 wt % to about 10 wt % the total composition of the liposomal payload;
d7) from about 4 wt % to about 10 wt % the total composition of the liposomal payload;
d8) from about 5 wt % to about 10 wt % the total composition of the liposomal payload; or
d9) from about 3 wt % to about 6 wt % the total composition of the liposomal payload.

Embodiments of the composition may comprise one or more liposomes comprising a payload, wherein the payload comprises concentrations of Thiamine as follows when the payload is in a fully dehydrated form:
f1) from about 0.1 wt % to about 2 wt % the total composition of the liposomal payload;
f2) from about 0.1 wt % to about 1.5 wt % the total composition of the liposomal payload;
f3) from about 0.1 wt % to about 1 wt % the total composition of the liposomal payload; or
f4) from about 0.3 wt % to about 1 wt % the total composition of the liposomal payload.

Embodiments of the composition may comprise one or more liposomes comprising a payload, wherein the payload comprises concentrations of Riboflavin as follows when the payload is in a fully dehydrated form:
g1) from about 0.1 wt % to about 2 wt % the total composition of the liposomal payload;
g2) from about 0.1 wt % to about 1.5 wt % the total composition of the liposomal payload;
g3) from about 0.1 wt % to about 1 wt % the total composition of the liposomal payload; or
g4) from about 0.3 wt % to about 1 wt % the total composition of the liposomal payload.

Embodiments of the composition may comprise one or more liposomes comprising a payload, wherein the payload comprises concentrations of Vitamin B-12 as follows when the payload is in a fully dehydrated form:
k1) from about 0.1 wt % to about 2 wt % the total composition of the liposomal payload;
k2) from about 0.1 wt % to about 1.5 wt % the total composition of the liposomal payload;
k3) from about 0.1 wt % to about 1.0 wt % the total composition of the liposomal payload;
k4) from about 0.1 wt % to about 0.9 wt % the total composition of the liposomal payload;
k5) from about 0.1 wt % to about 0.8 wt % the total composition of the liposomal payload;
k6) from about 0.1 wt % to about 0.7 wt % the total composition of the liposomal payload;
k7) from about 0.1 wt % to about 0.6 wt % the total composition of the liposomal payload;
k8) from about 0.1 wt % to about 0.5 wt % the total composition of the liposomal payload; or
k9) from about 0.1 wt % to about 0.4 wt % the total composition of the liposomal payload.

Embodiments of the composition may comprise one or more liposomes comprising a payload, wherein the payload comprises concentrations of magnesium as follows when the payload is in a fully dehydrated form:
p1) from about 2 wt % to about 10 wt % the total composition of the liposomal payload;
p2) from about 2 wt % to about 9 wt % the total composition of the liposomal payload;
p3) from about 2 wt % to about 8 wt % the total composition of the liposomal payload;
p4) from about 2 wt % to about 7 wt % the total composition of the liposomal payload;
p5) from about 2 wt % to about 6 wt % the total composition of the liposomal payload;
p6) from about 3 wt % to about 6 wt % the total composition of the liposomal payload;
p7) from about 4 wt % to about 6 wt % the total composition of the liposomal payload; or
p8) from about 5 wt % to about 6 wt % the total composition of the liposomal payload.

Embodiments of the composition may comprise one or more liposomes comprising a payload, wherein the payload comprises concentrations of zinc as follows when the payload is in a fully dehydrated form:
q1) from about 0.5 wt % to about 5 wt % the total composition of the liposomal payload;
q2) from about 0.5 wt % to about 4 wt % the total composition of the liposomal payload;
q3) from about 1 wt % to about 5 wt % the total composition of the liposomal payload;
q4) from about 2 wt % to about 5 wt % the total composition of the liposomal payload;
q5) from about 3 wt % to about 5 wt % the total composition of the liposomal payload; or
q6) from about 3 wt % to about 4 wt % the total composition of the liposomal payload.

Embodiments of the composition may comprise one or more liposomes comprising a payload, wherein the payload comprises concentrations of manganese as follows when the payload is in a fully dehydrated form:
s1) from about 0.1 wt % to about 2 wt % the total composition of the liposomal payload;
s2) from about 0.2 wt % to about 2 wt % the total composition of the liposomal payload;
s3) from about 0.3 wt % to about 2 wt % the total composition of the liposomal payload; or
s4) from about 0.3 wt % to about 1 wt % the total composition of the liposomal payload.

The foregoing percentages, concentrations, and ratios are presented by example only and are not intended to be exhaustive or to limit the disclosure to the precise percentages, concentrations, and ratios disclosed. It should be appreciated that each value that falls within a disclosed range is disclosed as if it were individually disclosed as set forth herein. For example, a range indicating a weight percent from about 8% to about 14% additionally includes ranges beginning or ending with all values within that range, including for example a range beginning at 8.1%, 8.2%, 8.3%, 9%, 10%, 11%, 12%, and so forth.

Also, according to one or more non-limiting embodiments of the disclosure, any of the concentrations for ingredients for a combination of the ingredients (a) through (pp), for example, as listed above, may indicate the concentration for other ingredients listed above.

The payload disposed within the liposome comprises water in addition to a plurality of different nutrients when the liposomal nutrient supplement is first manufactured. The liposomal nutrient supplement is later dehydrated such that it can be delivered to a user in a powdered form. The relative mass percentages described above measure the relative concentrations of the plurality of different nutrients within the payload without factoring in the water content. It should be appreciated that the liposomal nutrient supplement may further comprise water disposed within the internal core of the payload.

The payload disposed within the internal core of the liposome and/or disposed anywhere within the liposome including the phospholipid bilayer comprises a plurality of different and unique nutrients, wherein the nutrients comprise one or more of a vitamin, mineral, plant extract, herb extract, fungus extract, enzyme, acid, or amino acid. The payload may include any combination of vitamins, minerals, plant extracts, fungi extracts, enzymes, acids, amino acids, or other nutrients.

The payload may comprise one or more active ingredients selected from a group comprising water soluble vitamins, fat soluble vitamins, minerals, herbs and specialty nutrients.

The nutrient payload may comprise water soluble vitamins from the group comprising: Vitamin B1 (thiamine, benfotiamine); Vitamin B2 (riboflavin, riboflavin-5-phosphate); Vitamin B3 (niacin, nicotinic acid); Vitamin B5 (pantothenic acid); Vitamin B6 (pyridoxine, pyridoxal, pyridoxamine, pyridoxine-5-phosphate); Vitamin B12 (cobalamin, methylcobalamin); Biotin; and Vitamin C (ascorbic acid).

The payload may comprise fat soluble vitamins from the group comprising: Vitamin A (retinol, retinal, retinyl esters, retinoic acid, beta carotene); Vitamin D (calciferol, D-3 cholecalciferol, D-2 ergocalciferol); Vitamin E (d-alpha tocopherol, tocotrienols, tocopherols); Vitamin K1 (phylloquinone, menadione); and Vitamin K-2 (MK-7).

The payload may comprise one or more vitamins selected from a group comprising: Vitamin A (retinol, retinal, retinyl esters, retinoic acid, beta carotene); Vitamin B1 (thiamine, benfothiamine); Vitamin B2 (riboflavin, riboflavin-5-phosphate); Vitamin B3 (niacin, nicotinic acid); Vitamin B5 (pantothenic acid); Vitamin B6 (pyridoxine, pyridoxal, pyridoxamine, pyridoxine-5-phosphate); Vitamin B12 (cobalamin, methylcobalamin); Biotin; Vitamin C (ascorbic acid); Vitamin D (calciferol); Vitamin E (alpha-tocopherol); Vitamin B9 (folic acid, folate, folacin, (6S)-5-Methyltetrahydrofolic Acid); and Vitamin K (phylloquinone, menadione).

The payload may comprise one or more minerals selected from a group comprising: Boron; Calcium; Chloride; Chromium; Copper; Iodine; Iron; Magnesium; Manganese; Molybdenum; Phosphorus; Potassium; Selenium; Sodium; Vanadium; and Zinc.

The payload may comprise one or more plant extracts, whole herbs, fungi or herb extracts selected from a group comprising: Aloe vera; Alpinia Galanga; Ashwagandha; Berberine; Bilberry; Bitter Orange; Black Cohosh; Cayenne; Cranberry; Echinacea; Eleuthero; Ginkgo biloba; Ginseng; Goldenseal; Grapefruit Seed Extract; Green Tea Extract; Guggul; Holy Basil; Kava Kava; Lion's Mane Mushroom; Mangosteen; Maca; Marshmallow; Milk Thistle; Nettle; Olive Leaf; Oregano Oil; Passion Flower; Pumpkin Seed; Rose Hips; Quercetin; Reishi Mushroom; Resveratrol; Saw Palmetto; Senna; Schizandra; Shiitake Mushroom; Slippery Elm; Tart Cherry; Turmeric; Yarrow; Yohimbe; and Vinpocetine. The plant extracts may comprise herbs and other plants.

The payload may comprise one or more amino acids selected from a group comprising: Alanine; Arginine; Asparagine; Aspartic Acid; Cysteine; Glutamic Acid; Glutamine; Glycine; Histidine; Isoleucine; Leucine; Lysine; Methionine; Phenylalanine; Proline; Serine; Threonine; Tryptophan; Tyrosine; Valine; and Selenocysteine.

The payload may comprise one or more enzymes or other specialty nutrients selected from a group comprising: Alpha Lipoic Acid; Astaxanthin; Chondroitin; Coenzyme Q10; Collagen; Glucosamine; Hyaluronic Acid; Lutein; Melatonin; Omega Fatty Acids (derived from both algae and fish); and Probiotics (spores, Lactobacillus and Bifidobacterium species).

The compositions described herein may include methylated B vitamins. Methylation is a type of reaction that occurs in cells within the body. Methylation facilitates processes involved in gene expression, energy production, recycling by-products, and detoxification. Vitamins must be converted to the methylated form to be used by the body. The liposomal delivery system described herein enables direct delivery of methylated vitamins that are already within the active form and can be quickly utilized by the body.

The compositions described herein include trace minerals. Trace minerals are essential nutrients that the body cannot produce and must come from diet. Trace minerals are required for numerous physiological functions. For example, trace minerals serve as building blocks for hundreds of enzymes, they are necessary for hormone product, they support cognitive function, they transport oxygen to cells, and they are necessary for the maintenance and repair of tissue and bone. The liposomal delivery systems described herein enable direct delivery and improved absorption of trace minerals.

In an embodiment, a liposomal nutrient supplement is prepared to improve whole-body health in a user. The payload disposed within the liposomal shell comprises an optimized combination of nutrients for improving whole-body health. Specifically, the nutrient payload may comprise an effective amount of Vitamin A to support healthy immune response and skin support. The payload may comprise an effective amount of thiamine for improving the body's ability to convert food into energy, and further for improving the growth and strength of skin, hair, muscles, and nerves. The payload may comprise an effective amount of riboflavin for improving the body's ability to convert food into energy, and further for improving the growth and strength of skin, hair, muscles, and nerves. The payload may comprise an effective amount of niacin for improving the body's ability to convert food into energy, and further for improving the growth and strength of skin, hair, muscles, and nerves. The payload may comprise an effective amount of pantothenic acid for improving the body's ability to convert food into energy, and further for improving the body's ability to generate lipids, neurotransmitters, steroid hormones, and hemoglobin.

Further, the payload may comprise an effective amount of pyridoxine for lowering homocysteine levels to support healthy cardiovascular function. The payload may comprise an effective amount of cobalamin for lowering homocysteine levels to support healthy cardiovascular function. The payload may comprise an effective amount of biotin for improving the body's ability to support healthy hair skin and nail formation and convert food into energy and synthesize glucose. The payload may comprise an effective amount of biotin to improve the growth and strength of bones and hair. The payload may comprise an effective amount of Vitamin C for improving the body's ability to produce collagen for healthy skin, joint and bone formation and support blood vessel walls. The payload may comprise an effective amount of Vitamin C for providing sufficient antioxidant activity in the body to reduce oxidation levels in the body.

Further, the payload may comprise an effective amount of choline for supporting the body in producing and releasing the neurotransmitter acetylcholine, which aids in nerve and brain activities. The payload may comprise an effective amount of choline for providing sufficient choline for metabolizing and transporting fats. The payload may comprise an effective amount of calciferol for maintaining normal blood levels of calcium and phosphorous. The payload may comprise an effective amount of alpha-tocopherol for providing sufficient antioxidants to optimize the body's oxidation levels and reduce oxidative stress. The payload may comprise an effective amount of alpha-tocopherol for protecting Vitamin A and certain lipids from damage. The payload may comprise an effective amount of folic acid for reducing the likelihood of brain and spine birth defects when taken during pregnancy. The payload may comprise an effective amount of folic acid for lowering levels of homocysteine to support healthy cardiovascular function. The payload may comprise an effective amount of phylloquinone menadione for activating proteins and calcium essential to blood clotting, and further for supporting strong, healthy bones, thereby reducing the likelihood of a bone fracture.

Further, the payload may comprise an effective amount of calcium for supporting the body in building and protecting bones and teeth. The payload may comprise an effective amount of chloride for balancing fluids in the body and enabling the body to produce sufficient stomach acid for digestion. The payload may comprise an effective amount of chromium for enhancing the activity of insulin and maintaining normal blood glucose levels. The payload may comprise an effective amount of copper for aiding in iron metabolism and increasing the production of red blood cells. The payload may comprise an effective amount of fluoride for increasing bone formation and reducing the likelihood of dental cavities. The payload may comprise an effective amount of iodine for improving thyroid function. The payload may comprise an effective amount of iron for improving oxygen transport by myoglobin and red blood cells. The payload may comprise an effective amount of magnesium for improving muscle contraction and blood clotting. The payload may comprise an effective amount of manganese for improving the metabolization of amino acids, cholesterol, and carbohydrates. The payload may comprise an effective amount of potassium for lowering blood pressure.

In an embodiment, a liposomal nutrient supplement is prepared specifically for women's health. This implementation comprises the vitamins and minerals necessary to support women's health. Specifically, this implementation may comprise thirty or more liposomal nutrients for whole body wellness, including 15 or more vitamins and minerals. The women's liposomal nutrient supplement comprises an effective amount of GABA to improve digestion function; an effective amount of ashwagandha extract to improve adrenal function; and/or an effective amount of biotin for supporting healthy hair and nail growth. The liposomal nutrient supplement further includes methylated B vitamins.

The women's liposomal nutrient supplement may specifically include a plurality of nutrients selected from a list comprising Vitamin A, Vitamin C, Vitamin D-3, Vitamin E, Vitamin K-1, Thiamine, Riboflavin, Niacin, Vitamin B-6, Folate, Vitamin B-12, Biotin, Pantothenic Acid, Choline, Iron, Iodine, Magnesium, Zinc, Selenium, Manganese, Chromium, Molybdenum, Glycine, Inositol, PABA, Liposomal Trace Mineral Complex, Lutein, Coenzyme Q10, Rose Hips, Acerola, Citrus Bioflavonoids, Silica, Aloe Extract, Ginger Extract, Ashwagandha Extract, GABA, and/or Hyaluronic Acid.

In an embodiment, a liposomal nutrient supplement is prepared specifically for men's health. This implementation comprises the vitamins and minerals necessary to support men's health. Specifically, this composition may include thirty or more liposomal nutrients for whole body wellness, including 15 or more vitamins and minerals. The men's liposomal nutrient supplement may specifically include branched-chain amino acids for muscle support and pumpkin seed extract for prostate health. The composition may further include methylated B vitamins.

The men's liposomal nutrient supplement may specifically include a plurality of nutrients selected from a list comprising Vitamin A, Vitamin C, Vitamin D-3, Vitamin E, Vitamin K-1, Thiamine, Riboflavin, Niacin, Vitamin B-6, Folate, Vitamin B-12, Biotin, Pantothenic Acid, Choline, Iodine, Magnesium, Zinc, Selenium, Manganese, Chromium, Molybdenum, Glycine, Inositol, PABA, Liposomal Trace Mineral Complex, Lutein, Coenzyme Q10, Rose Hips, Acerola, L-Leucine, L-Isoleucine, L-Valine, L-glutamine, and Pumpkin Seed Extract.

In an embodiment, a liposomal nutrient supplement is prepared specifically for women's health over 50 years of age. This implementation comprises the vitamins and minerals necessary to support women's health in advanced age after 50 years of age. Specifically, this implementation may comprise thirty or more liposomal nutrients for whole body wellness, including 15 or more vitamins and minerals. The women's 50+ liposomal nutrient supplement comprises an effective amount of biotin to increase the growth and strength of hair and nails; and an effective amount of cranberry extract to support healthy urinary tract function. The liposomal nutrient supplement for women over age 50 further includes methylated B vitamins.

The women's age 50 and over liposomal nutrient supplement may specifically include a plurality of nutrients selected from a list comprising Vitamin A, Vitamin C, Vitamin D-3, Vitamin E, Vitamin K-1, Thiamine, Riboflavin, Niacin, Vitamin B-6, Folate, Vitamin B-12, Biotin, Pantothenic Acid, Choline, Iron, Magnesium, Zinc, Selenium, Manganese, Chromium, Molybdenum, Vitamin K-2, Glycine, Inositol, PABA, Trace Mineral Complex, Lutein, Coenzyme Q10, Rose Hips, Acerola, Aloe Extract, Ginger Extract, Ashwagandha Extract, and Cranberry Extract.

In an embodiment, a liposomal nutrient supplement is prepared as a universal formula to support wholistic health in all adults. This implementation comprises the vitamins and minerals necessary to support whole body wellness. Specifically, this implementation may comprise thirty or more liposomal nutrients for whole body wellness, including 15 or more vitamins and minerals. The liposomal nutrient supplement includes methylated B vitamins.

The universal adult liposomal nutrient supplement may specifically include a plurality of nutrients selected from a list comprising Vitamin A, Vitamin C, Vitamin D-3, Vitamin E, Vitamin K-1, Thiamine, Riboflavin, Niacin, Vitamin B-6, Folate, Vitamin B-12, Biotin, Pantothenic Acid, Choline, Iron, Iodine, Magnesium, Zinc, Selenium, Manganese, Chromium, Molybdenum, Glycine, Inositol, PABA, Liposomal Trace Mineral Complex, Lutein, Coenzyme Q10, Rose Hips, and Acerola.

In an embodiment, the liposomal nutrient supplement described herein is dehydrated. The dehydrated liposomal nutrient supplement is provided to a user in a powdered form. Powder liposomes are dehydrated and are more stable in supplements. Clinical studies indicate that powdered liposomes withstand gastric acid and six-times better absorbed than liquid liposomes.

Example 1 is a composition. The composition includes a liposome comprising a lipid bilayer, wherein the liposome comprises an internal core. The composition includes a payload disposed within the liposome. The composition is such that the payload comprises a plurality of different supplement nutrients comprising one or more of a vitamin, mineral, extract, enzyme, or amino acid.

Example 2 is a composition as in Example 1, wherein the plurality of different nutrients comprises vitamin A, folate, magnesium, selenium, zinc, thiamine, riboflavin, and niacin.

Example 3 is a composition as in any of Examples 1-2, wherein the liposome is dehydrated, and wherein the composition is provided to a user in a powdered form.

Example 4 is a composition as in any of Examples 1-3, wherein the dehydrated liposome is configured to be rehydrated in a gastrointestinal tract of the user, and wherein at least a portion of the payload is absorbed into a bloodstream of the user.

Example 5 is a composition as in any of Examples 1-4, wherein the payload comprises an effective amount of biotin for increasing speed of hair and nail growth in a user, and further an effective amount of the biotin for increasing strength of hair and nails in the user.

Example 6 is a composition as in any of Examples 1-5, wherein the payload comprises a plurality of hydrophilic substances and further comprises a plurality of hydrophobic substances.

Example 7 is a composition as in any of Examples 1-6, wherein, during manufacturing of the composition, the payload further comprises water, and wherein the composition is dehydrated prior to delivery to a user.

Example 8 is a composition as in any of Examples 1-7, wherein the composition is a liposomal nutrient supplement, and wherein the payload of the liposomal nutrient supplement is optimized for improving women's health and comprises: an effective amount of iron for improving muscle metabolism; an effective amount of hyaluronic acid for supporting joint lubrication; and an effective amount of ashwagandha extract for supporting adrenal function.

Example 9 is a composition as in any of Examples 1-8, wherein the composition is a liposomal nutrient supplement, and wherein the payload of the liposomal nutrient supplement is optimized for improving men's health and comprises: an effective amount of branched chain amino acids for improving muscle growth; and an effective amount of pumpkin seed extract for improving prostate function.

Example 10 is a composition as in any of Examples 1-9, wherein the composition is a liposomal nutrient supplement, and wherein the payload of the liposomal nutrient supplement is optimized for improving women's health over the age of 50 and comprises: an effective amount of cranberry extract for supporting healthy urinary tract function; and an effective amount of vitamin K2 for improving bone metabolism.

Example 11 is a composition as in any of Examples 1-10, wherein the composition is a liposomal nutrient supplement, and wherein the payload of the liposomal nutrient supplement is optimized for improving whole body wellness in an adult and comprises vitamin A, vitamin C, vitamin E, vitamin B-6, vitamin B-12, thiamine, riboflavin, niacin, folate, biotin, choline, magnesium, zinc, selenium, manganese, molybdenum, and coenzyme Q10.

Example 12 is a composition as in any of Examples 1-11, wherein the payload comprises one or more of: methylated vitamin B-2; methylated vitamin B-6; or methylated vitamin B-12.

Example 13 is a composition as in any of Examples 1-12, wherein the composition is dehydrated and disposed in a capsule, and wherein the capsule is prepared for ingestion by a user.

Example 14 is a composition as in any of Examples 1-13, further comprising one or more of cellulose, magnesium stearate, stearic acid, tapioca starch, silica, sodium alginate, pea starch, or gum arabic.

Example 15 is a composition as in any of Examples 1-14, wherein the lipid bilayer comprises lipids isolated from sunflower lecithin.

Example 16 is a composition as in any of Examples 1-15, wherein the payload comprises vitamin A, and wherein the vitamin A comprises a concentration from about 0.1 wt % to about 2 wt % a total composition of the payload.

Example 17 is a composition as in any of Examples 1-16, wherein the payload comprises vitamin C, and wherein the vitamin C comprises a concentration from about 15 wt % to about 95 wt % a total composition of the payload.

Example 18 is a composition as in any of Examples 1-17, wherein the payload comprises vitamin E, and wherein the vitamin E comprises a concentration from about 2 wt % to about 10 wt % a total composition of the payload.

Example 19 is a composition as in any of Examples 1-18, wherein the payload comprises thiamine and riboflavin, and wherein: the thiamine comprises a concentration from about 0.1 wt % to about 2 wt % a total composition of the payload; and the riboflavin comprises a concentration from about 0.1 wt % to about 2 wt % the total composition of the payload.

Example 20 is a composition as in any of Examples 1-19, wherein the payload comprises each of vitamin A, vitamin C, vitamin D-3, vitamin E, vitamin K-1, thiamine, riboflavin, vitamin B-6, folate, vitamin B-12, biotin, choline, iodine, magnesium, zinc, selenium, manganese, chromium, molybdenum, glycine, inositol, coenzyme Q10, and vitamin B10.

Example 21 is a composition as in any of Examples 1-20, wherein the plurality of different nutrients of the payload comprises at least one of each of the vitamin, the mineral, the plant extract, and the amino acid.

Example 22 is a composition as in any of Examples 1-21, wherein the plurality of different nutrients of the payload comprises a plurality of different vitamins, a plurality of different minerals, one or more plant extracts, and one or more amino acids.

Example 23 is a composition as in any of Examples 1-22, wherein the composition is dehydrated such that the composition comprises from about 0 wt % to about 30 wt % water by mass of the total composition.

Example 24 is a composition as in any of Examples 1-23, wherein the composition is provided to a user in a dehydrated powdered form, wherein the dehydrated powder is disposed within a capsule that is dissolvable by the user's gastrointestinal system.

Example 25 is a composition as in any of Examples 1-24, wherein the composition is prepared for oral delivery to a user.

Example 26 is a composition as in any of Examples 1-25, wherein the oral delivery may include solid dose vehicles such as capsules and tablets.

Example 27 is a composition as in any of Examples 1-26, wherein the oral delivery further includes powders, gummies, softgels, liquids, mouth strips and liquid capsules.

Example 28 is a composition as in any of Examples 1-27, wherein the composition is prepared for topical delivery to a user.

Example 29 is a composition as in any of Examples 1-28, wherein the lipid bilayer comprises one or more lecithins.

Example 30 is a composition as in any of Examples 1-29, wherein the lipid bilayer is a phospholipid bilayer comprises a lecithin extracted from one or more of soybean, sunflower, canola, egg yolk, or algae.

Example 31 is a composition as in any of Examples 1-30, wherein the liposome is prepared using a high-shear processing method to break down particle size.

Example 32 is a composition as in any of Examples 1-31, wherein the high-shear processing method comprises one or more of a freeze-thaw cycle, rotor-stator dispersion, sonication, extrusion, or high-pressure homogenizations.

Example 33 is a composition as in any of Examples 1-32, wherein a dimension and geometry of the liposome is optimized to increase kinetics and uptake by a body.

Example 34 is a composition as in any of Examples 1-33 wherein the lipid bilayer of the liposome attaches to a lipid bilayer of a cell within a user, and wherein the payload is absorbed by the cell within the user after the lipid bilayer of the liposome attaches to the lipid bilayer of the cell.

Example 35 is a composition as in any of Examples 1-34, wherein at least 5% of the plurality of the different nutrients of the payload are absorbed by a user and taken up by the user's bloodstream.

Example 36 is a composition as in any of Examples 1-35, wherein from about 2% to about 10% of the plurality of the different nutrients of the payload are absorbed by a user and taken up by the user's bloodstream.

Example 37 is a composition as in any of Examples 1-36, wherein from about 5% to about 40% of the plurality of the different nutrients of the payload are absorbed by a user and taken up by the user's bloodstream.

Example 38 is a composition as in any of Examples 1-37, wherein the payload is disposed within the internal core of the liposome.

Example 39 is a composition as in any of Examples 1-38, wherein the payload is disposed within the liposome including the lipid bilayer.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A composition comprising:
a liposome comprising a lipid bilayer, wherein the liposome comprises an internal core; and
a payload disposed within the liposome;
wherein the payload comprises a plurality of different supplement nutrients comprising one or more of a vitamin, mineral, herb, extract, fish oil, enzyme, probiotic, amino acid, or specialty nutrients.

2. The composition of claim 1, wherein the plurality of different nutrients comprises water soluble vitamins, fat soluble vitamins, minerals and herbs.

3. The composition of claim 1, wherein the liposome is dehydrated, and wherein the composition is provided to a user in a powdered form.

4. The composition of claim 3, wherein the dehydrated liposome is configured to be rehydrated in a gastrointestinal tract of the user, and wherein at least a portion of the payload is absorbed into a bloodstream of the user.

5. The composition of claim 1, wherein the payload further comprises water, and wherein the composition is provided to a user in an aqueous form.

6. The composition of claim 1, wherein the payload comprises a plurality of hydrophilic substances and further comprises a plurality of hydrophobic substances.

7. The composition of claim 1, wherein, during manufacturing of the composition, the payload further comprises water, and wherein the composition is dehydrated prior to delivery to a user.

8. The composition of claim 1, wherein the composition is a liposomal nutrient supplement, and wherein the payload of the liposomal nutrient supplement is optimized for improving women's health and comprises:
an effective amount of iron for improving healthy blood cell formation;
an effective amount of hyaluronic acid for improving skin and joint lubrication; and
an effective amount of ashwagandha extract for improving vagal nerve and adrenal function.

9. The composition of claim 1, wherein the composition is a liposomal nutrient supplement, and wherein the payload of the liposomal nutrient supplement is optimized for improving men's health and comprises:
an effective amount of branched chain amino acids for improving muscle growth; and
an effective amount of pumpkin seed extract for improving prostate function.

10. The composition of claim 1, wherein the composition is a liposomal nutrient supplement, and wherein the payload of the liposomal nutrient supplement is optimized for improving women's health over the age of 50 and comprises:
an effective amount of cranberry extract for supporting a healthy urinary tract; and
an effective amount of vitamin K2 for supporting healthy bone formation.

11. The composition of claim 1, wherein the composition is a liposomal nutrient supplement, and wherein the payload of the liposomal nutrient supplement is optimized for improving whole body wellness in an adult and comprises vitamin A, vitamin C, vitamin E, vitamin B-6, vitamin B-12, thiamine, riboflavin, niacin, folate, biotin, choline, magnesium, zinc, selenium, manganese, molybdenum, and coenzyme Q10.

12. The composition of claim 1, wherein the payload comprises one or more of:
methylated vitamin B-2;
methylated vitamin B-6; or
methylated vitamin B-12.

13. The composition of claim 1, wherein the composition is dehydrated and disposed in a capsule, and wherein the capsule is prepared for ingestion by a user.

14. The composition of claim 1, further comprising one or more of cellulose, magnesium stearate, stearic acid, tapioca starch, silica, sodium alginate, pea starch, or gum arabic.

15. The composition of claim 1, wherein the lipid bilayer comprises lipids isolated from sunflower lecithin.

16. The composition of claim 1, wherein the payload comprises vitamin A, and wherein the vitamin A comprises a concentration from about 0.1 wt % to about 2 wt % a total composition of the payload.

17. The composition of claim 1, wherein the payload comprises vitamin C, and wherein the vitamin C comprises a concentration from about 15 wt % to about 95 wt % a total composition of the payload.

18. The composition of claim 1, wherein the payload comprises vitamin E, and wherein the vitamin E comprises a concentration from about 2 wt % to about 10 wt % a total composition of the payload.

19. The composition of claim 1, wherein the payload comprises thiamine and riboflavin, and wherein:
the thiamine comprises a concentration from about 0.1 wt % to about 2 wt % a total composition of the payload; and
the riboflavin comprises a concentration from about 0.1 wt % to about 2 wt % the total composition of the payload.

20. The composition of claim 1, wherein the payload comprises each of vitamin A, vitamin C, vitamin D-3, vitamin E, vitamin K-1, thiamine, riboflavin, vitamin B-6, folate, vitamin B-12, biotin, choline, iodine, magnesium, zinc, selenium, manganese, chromium, molybdenum, glycine, inositol, coenzyme Q10, and vitamin B10.
